(19) United States
(12) Patent Application Publication
(10) Pub. No.: US 2005/0004632 A1
(43) Pub. Date: Jan. 6, 2005

(54) Universal light processing for a human body

(76) Inventor: Mellen-Thomas Benedict, Aptos, CA (US)

Correspondence Address: SCHNECK & SCHNECK, P.O. BOX 2-E, SAN JOSE, CA, 95109-0005, US

(21) Appl. No.: 10/370,330

(22) Filed: Feb. 18, 2003

Related U.S. Application Data
(63) Continuation-in-part of application No. 09/803,079, filed on Mar. 8, 2001, now Pat. No. 6,524,329.

(51) Int. Cl. A61N 5/06

(52) U.S. Cl. 607/88; 607/91

(57) ABSTRACT

A system for illuminating selected body components of, or all of, a human body. A recliner apparatus is provided with a body support surface and a canopy that rotates over a portion of the body. A plurality of spaced apart light sources (near-uv, visible, near-ir) and/or spaced apart low frequency wave sources and/or spaced apart magnetic field sources is located on or adjacent to the body support surface and/or canopy to provide intermittent or continuous illumination of selected body components. The light sources provide two, three or more different wavelength ranges, in time intervals spaced apart by dark field time intervals. The recliner apparatus has a plurality of linear and curvilinear shapes and a shape adjustment mechanism that adjusts the recliner shape between sessions or during a session.

### FIELD OF THE INVENTION

This invention relates to illumination of a body, using light with selected wavelength ranges and selected illumination time intervals.

### BACKGROUND OF THE INVENTION

Phototherapy involves generation of light by suitable light sources, such as light emitting diodes (LEDs) in the visible and infrared ranges to provide various benefits for a patient's body. The photons produced are absorbed by the body through the skin, the eyes and acupuncture points or meridians. Connective tissues in the body conduct the light to deeper tissues and organs. By taking advantage of optical properties of biological tissues, suitable wavelengths of light can be delivered to, absorbed by and used by the body to activate metabolic functions.

Treatment of a body using light irradiation requires a choice of several important parameters, including wavelength range, relative distribution of the wavelengths within the range (spectrum), time interval for continuous exposure, time interval between two continuous exposures, time rate of energy delivered, accumulated energy density for exposures, body component(s) irradiated, and many others. In some instances, different parts of the body require different light processing parameters.

What is needed is a method and corresponding system that provides appropriate illumination for a whole body that optionally allows a choice of different relevant light processing parameters for different body components and that distinguishes between treatments for different medical purposes. Preferably, the method and system should provide for, and distinguish between, initial treatments and maintenance treatments for a given medical condition and should cover a large number of, if not all of, conditions that are believed to be treatable using illumination.

### SUMMARY OF THE INVENTION

These needs are met by the invention, which provides a system that applies radiation in selected wavelength ranges to a whole body, using a controlled sequence of exposures that optionally illuminates different components of the body using different electromagnetic processing parameters. Any two consecutive time intervals of continuous radiation exposure in a selected wavelength range are spaced apart by a “dark field” time interval whose length is at least equal to a threshold value, in order to re-establish a randomization of electron transport and distribution resulting from application of photons during a continuous exposure interval. The system user or a consultant selects a preferred illumination schedule for a session, including specification of one or more of the following parameters: (1) the whole body or specified body components to be illuminated; (2) temporal length of the session; (3) the intensity(ies) of light to be delivered by each light source in the light delivery system; (4) the wavelength range(s) of light to be delivered to the whole body or to specified body components; (5) the exposure time Δt(exp) for each wavelength group used; (6) the dark time interval length Δt(dark) for each wavelength range used; (7) the light energy delivery rate to be delivered to the whole body or to specified body components; (8) the accumulated time light is to be delivered to the whole body or to specified body components; (9) the intensity(ies) of the magnetic field sources; (10) the frequency(ies) (including 0 Hz or dc) for the magnetic field sources; (11) the intensity(ies) of the LF frequency sources; (12) the frequency(ies) for the LF frequency sources; (13) the intensity(ies), radio frequency(ies) and time interval(s) of application of the radio waves; and (14) one or more shape parameters for the body support surface for one or more parts of a session.

The user positions himself or herself in a recliner that includes one or more arrays of illumination devices located adjacent to one or more of: (1) the foot and ankle area(s); (2) the lower leg area(s); (3) the upper leg area(s); (4) the hip and lower torso area(s); (5) the upper torso area(s); (6) the shoulder and upper arm area(s); (7) the lower arm and wrist area(s); (8) the hand area(s); (9) the neck and shoulder(s) area; (10) the lower head area; and (11) the upper head area. One or more of these areas can be targeted in isolation, or several or all areas can be targeted simultaneously or sequentially. After the user is positioned in the recliner, the illumination system is activated, in accord with the user's choice of schedule parameters.

Radiation is delivered to the whole body, or to the specified body components, using an enhanced focussing system that increases the efficiency of delivery of the radiation. The radiation delivery system can be fitted or molded to preferentially illuminate only the specified body components, if desired. Several different modules are provided, including light delivery components that can be combined or used in stand-alone mode for delivery of light to part or all of the head, or to one or more other selected body parts and/or one or more selected acupuncture sites. Light therapy in the visible range, the near-infrared range and/or the near ultraviolet range can be combined radio waves and with static or time-varying magnetic fields, using the same or a separately specified schedule, to provide additional effects and benefits.

### BRIEF DESCRIPTION OF THE DRAWINGS

FIG. 1 schematically illustrates an embodiment of recliner apparatus, for delivery of radiation to the whole body or to specified body components.

FIG. 2 illustrates a format for a control pattern suitable for entering a schedule for illumination of the whole body or of specified body components.

FIGS. 3A and 3B illustrate use of light delivery wrap mechanisms.

FIGS. 4, 5 and 6 schematically illustrate suitable patterns of light sources for different wavelengths.

FIGS. 7A and 7B graphically illustrate time intervals for irradiation using different wavelength ranges according to two embodiments of the invention.

FIGS. 8, 9 and 10 illustrate suitable light intensity patterns versus time for delivery of radiation according to the invention.

FIG. 11 is a representative graphical view of an average number of free electrons produced by an incident photon with a specified energy E.

FIGS. 12A-12F illustrate suitable cross sectional shapes of the recliner body support.

### DESCRIPTION OF BEST MODES OF THE INVENTION

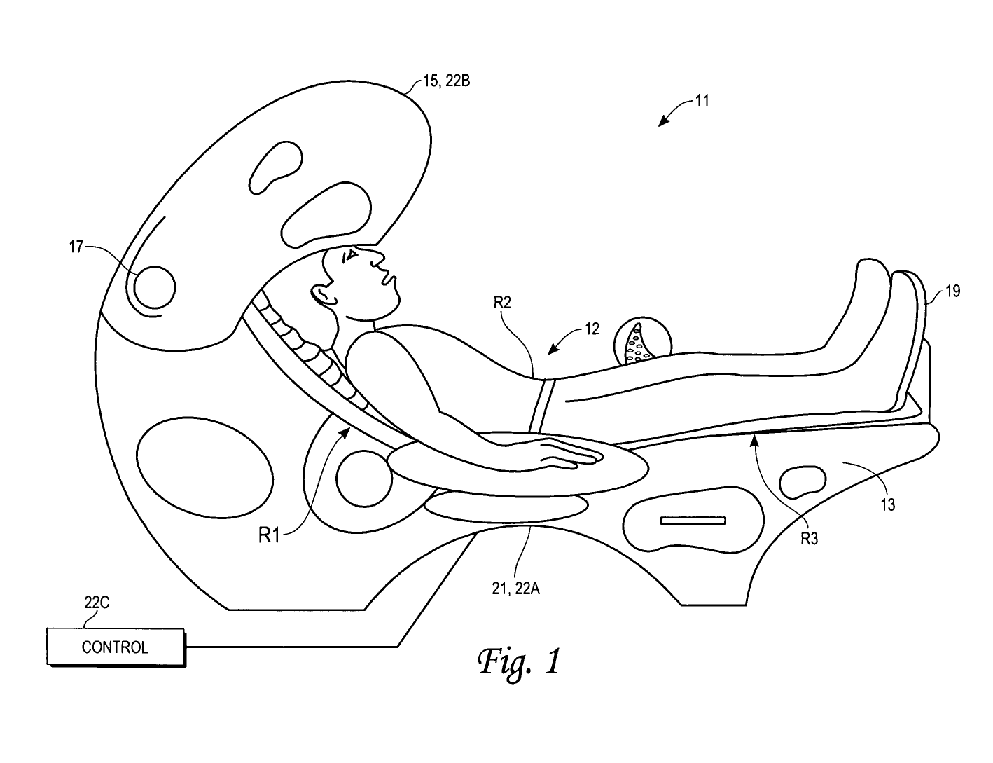

FIG. 1 illustrates a recliner system 11, configured to receive the body 12 of a user or patient as shown. The system 11 includes: a substantially horizontal body support module 13, having an adjustable curvilinear cross section to accommodate the user's body in a reclining or seated position and to provide access for irradiation of one or more selected body components; a rotatable hood module or canopy 15 that envelops an upper portion of the user's body 12, when the user reclines on the apparatus; the hood module rotates around one or more hinge or rotator assemblies 17, to allow the user to move onto, and to move off of, the support module 13 without undue bending or crouching; one or two foot modules 19, to receive the user's foot or feet, to help position the user's body 12 on the support module 13, and to deliver radiation thereto; and one or two arm modules 21, to receive and position the user's arm or arms and to deliver radiation thereto.

Each of the hood module 15, the foot module 19 and the arm module 21 has a plurality of radiation sources to deliver time varying radiation in one or more selected wavelength bands to a targeted portion of the user's body 12. The hood module 15, each foot module 19 and each arm module 21, is preferably divided into two or more independently activatable sub-modules, arranged to deliver radiation to an adjacent targeted portion of the body when one portion is to be irradiated and adjacent portions of the body are to be left substantially un-irradiated. In one embodiment, a control system 22A for the apparatus is located on or adjacent to one or both arm modules 21 so that the user can activate and deactivate the radiation delivery system and can change one or more schedule parameters associated with delivery of radiation to the user's body (location of target portion(s), radiation energy delivery rate, wavelength band(s) for radiation, length of a “dark time interval” between successive irradiation intervals, total exposure time for the target portion(s), etc.). In another embodiment, a control system 22B is located on the upper side or on the under side of the hood module 15. In another embodiment, a control system 22C is spaced apart from the recliner system 11.

The recliner system 11 can be supplemented by use of one or more radiation delivery body wrap modules, configured to be wrapped around, and to controllably deliver radiation to, a hand, a wrist, a lower arm, an elbow, an upper arm, a shoulder, a neck, an upper torso, a lower torso, an upper leg, a knee, a lower leg, an ankle, a foot and/or selected regions of a user's head. The system 11 shown in FIG. 1 can be supplemented with one or more body wrap modules to controllably direct radiation to a particular body part, or one or more body warp modules can be used by itself, without activation of the system 11, as illustrated in FIG. 4.

Low frequency (LF) wave sources, R1, R2 and R3 are positioned at three or more spaced apart locations on or associated with the recliner system 11 to provide intermittent or continuous LF illumination (including but not limited to ultrasound) that can reach more deeply into the body of a user who reclines on the system. A given region of the user's body need not be directly exposed to the LF waves (no direct line of sight is required), because almost any substance except heavy metals (having a high number of protons in the nucleus) and their alloys is transparent to an LF wave. Experiments indicate that as few as three LF wave sources suffice to bathe the user's body in adequate LF waves, but a greater number can be provided on the system 11, if desired. The frequencies of the LF waves have a preferred range of 1-10⁴ Hz and an accumulated intensity range of 0.1-20 Joules/cm².

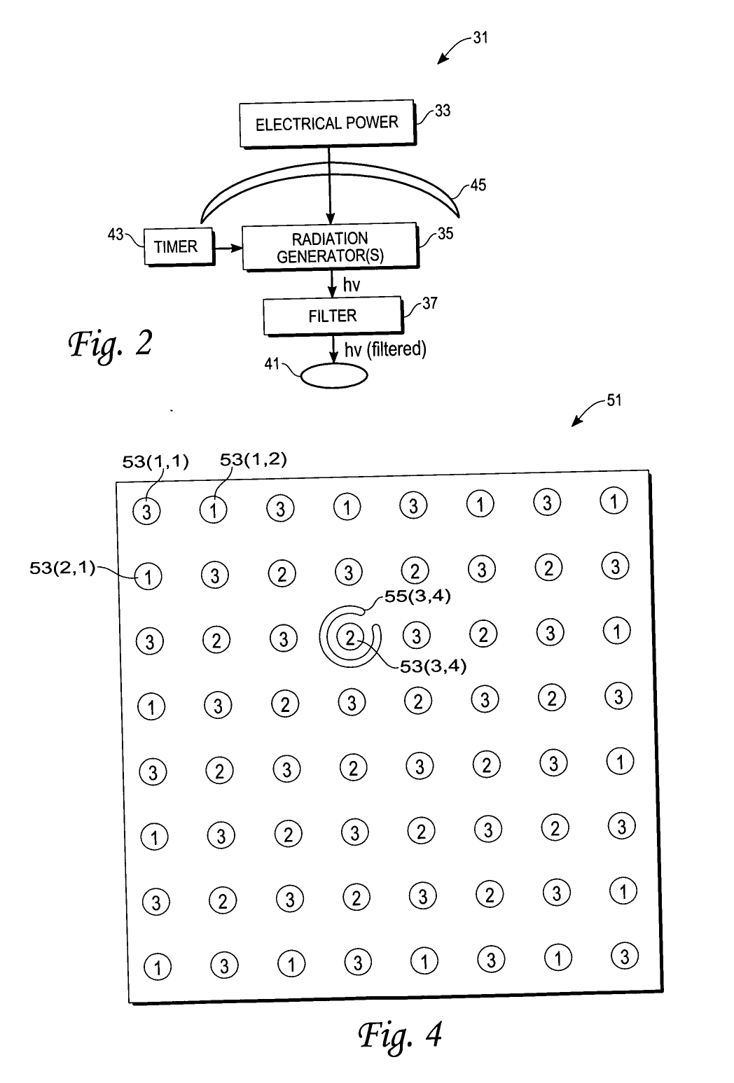

FIG. 2 illustrates a light delivery system 31 suitable for generating and delivering radiation to one or more selected body components according to the invention. The system 31 includes an electrical power source 33 that delivers controllable power to an assembly 35 of electromagnetic radiation generators, preferably to provide light in the visible and near infrared ranges (e.g., with wavelengths λ in a range 400 nm≦λ≦1500 nm). Optionally, the light generated by the radiation generator assembly 35 also may have wavelengths in a near-ultraviolet range (e.g., 350 nm≦λ≦400 nm) and may have longer wavelengths in a mid-infrared range (λ>1500 nm), or in selected portions of one or more of these wavelength ranges. For example, the wavelengths λ≈470 nm (useful for treating Alzheimer's disease), λ≈550 nm, λ≈637 nm, λ≈666 nm, λ≈890 nm and λ≈905 nm are useful for many treatments

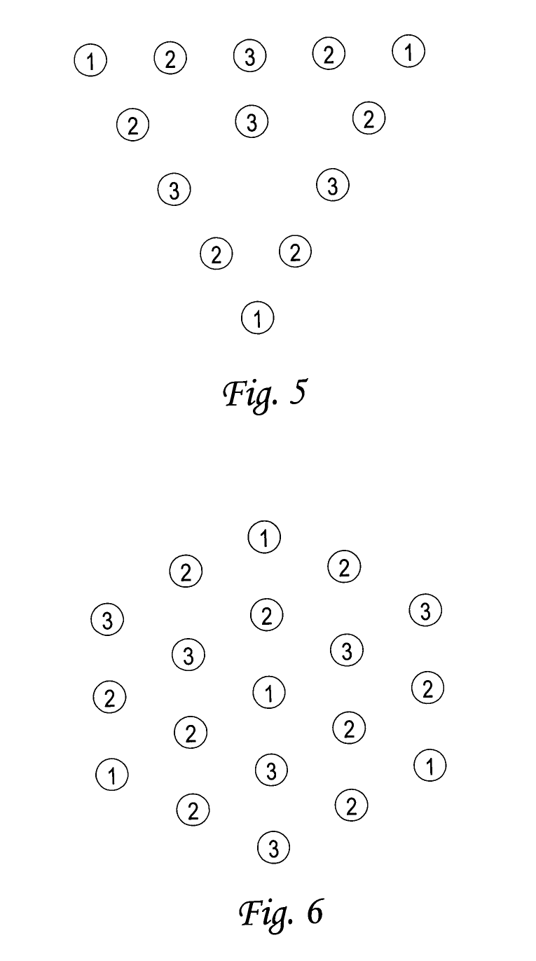

Each radiation generator in the assembly 35 may be a laser, a light emitting diode (LED), an intense incandescent light source, an intense fluorescent light source or any other suitable light source with optionally controllable light intensity, or a combination of two or more such light sources. LEDs have been and are being developed that can provide two, three or more different wavelength ranges from a single (multicolor) LED. For example, infrared, red, green, blue and/or white colors can be provided by changing one or more of the LED input parameters of a driving signal. Where an array of multicolor LEDs is used, each LED in the array may be driven by different LED drive signals at different times so that provision of two or more interleaved arrays, as suggested in FIGS. 4, 5 and 6, is not necessary.

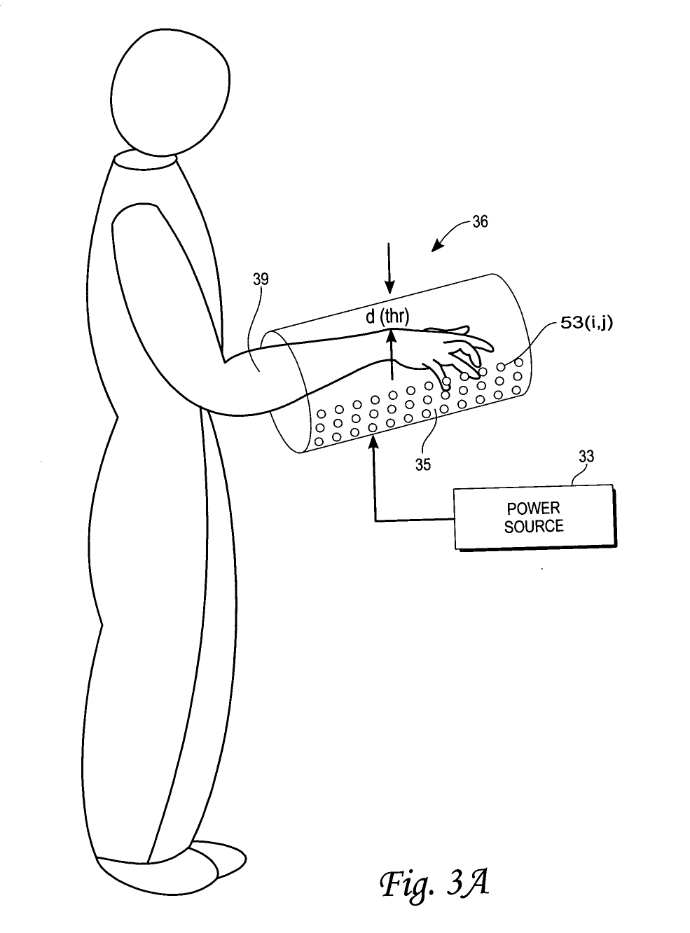

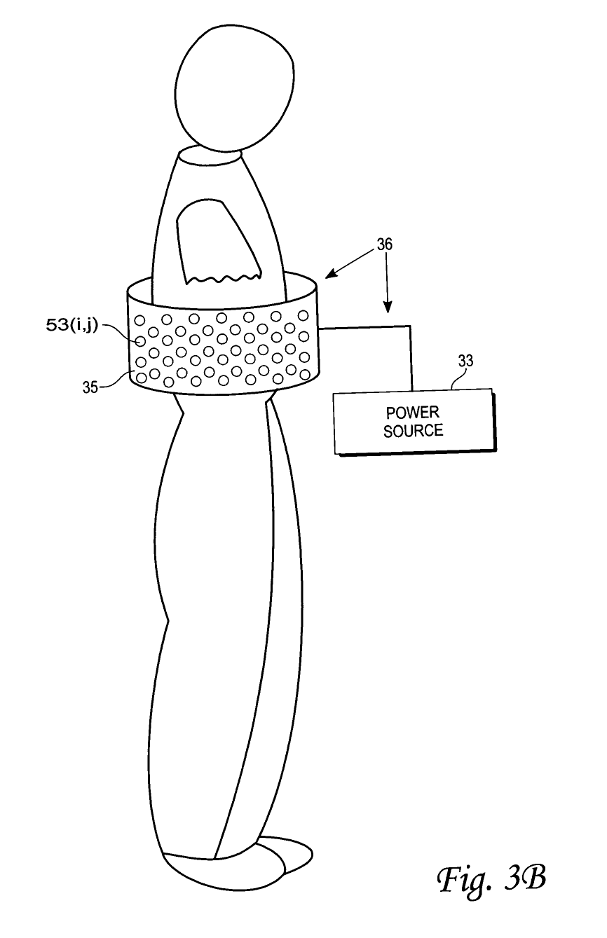

The radiation generator assembly 35 in FIG. 2 may be positioned on a light delivery wrap mechanism 36 (shown in an example in FIG. 3A, enveloping an arm and hand of a user, and in FIG. 3B, enveloping the lower back and lower torso of a user) that is configured to contact and wrap around a selected body component 39, a group of two or more adjacent body components or the whole body, so that each radiation generator is spaced apart from the body component 39 by at least a selected threshold standoff distance d(thr), to provide some control over the rate at which light is delivered to this body component. A suitable threshold standoff distance is d(thr)=1-15 cm. However, direct contact with the body is appropriate in some instances. If the assembly 35 provides light in one or more unwanted wavelength ranges, one or more filters 37 (optional) is positioned between the radiation generator assembly 35 and the selected body component(s) 39 to be treated. The radiation generator assembly 35 may produce a single beam or a few beams of light that are directed toward the body component 39, considered as a target. Preferably, the radiation generator assembly 35 produces many light beams that are directed toward the body component 39.

The system optionally includes a light focussing mechanism 41 that preferentially directs light produced by the radiation generator assembly 35 toward one or more target sites. In some situations, the light beams are produced in a pattern surrounding a selected body part, such as an arm or a leg, so that the selected body part and adjacent body parts are irradiated together in a (diffuse) field effect.

The radiation generator assembly 35 includes a timer 43 that activates and deactivates (turns on and turns off) individual radiation generators 53(i,j) during selected exposure time intervals, with any two consecutive continuous exposure (light) time intervals for a given wavelength having a first selected length Δt(exp), separated by a dark field time interval that has a second selected length Δt(dark). This (light/dark/light) activity and its inverse, (dark/light/dark), are sometimes referred to as a “reciprocating chase.” The first selected time interval length lies in a preferred range, 0.1 sec≦Δt(exp)≦1 sec, and the second selected time interval length Δt(dark) is preferably between 0.1 sec and 1 sec.

One or more light reflecting mechanisms 45 (optional) are positioned adjacent to the radiation generator assembly 35 to capture and direct light toward the selected body component 39 to couple some or all of the generated light, which would otherwise have been lost, into that body component. The light concentrator, condenser or other light focussing mechanism 45 is positioned between the radiation generator assembly 35 and the body component 39, to selectably concentrate (or to scatter within the body) the generated light on and around the body component 39, the whole body or selected sites on the selected body component.

FIGS. 4, 5 and 6 illustrate suitable polygonal light delivery patterns (rectangular, triangular and hexagonal, respectively) in which selected light sources (e.g., light emitting diodes) deliver light in one, two, three or more selected wavelength ranges. In FIG. 4, for example, the second row of the array 51 of light sources 53(i,j), with i=2, delivers light in the respective wavelengths ranges 1, 3, 2, 3, 2, 3, 2, 3.

Each light delivery element (e.g., 53(i,j) in FIG. 4) may deliver light in one or more selected wavelength ranges, when this element is activated, and adjacent light delivery elements may deliver the same, or different, wavelength ranges, chosen according to the treatment or therapy to be provided for adjacent body components. The chosen range of color(s) can be changed as a treatment or therapy session proceeds. In a preferred embodiment, each light delivery element, such as 53(i,j) in FIG. 4, delivers one or more selected ranges of light wavelengths. More generally, light in any of N color ranges can be delivered (e.g., N=7), and the color ranges are chosen and changed according to the treatment or therapy to be provided.

Optionally, a magnetic field element and/or a radio wave element 55(i,j) can be positioned adjacent to one or more of the light sources 53(i,j) to deliver a constant or time varying magnetic field to adjacent body components, as illustrated in FIG. 4. The frequencies used may be the same or may be different. The peak magnetic field can be 100-10⁴ Gauss, or greater if desired, and the frequency for a time varying magnetic field can be 1-10⁴ Hz, or greater if desired. The magnetic field vector (B or H) can be fixed in direction, or the vector direction can vary with time, and the field is optionally applied in two or more time intervals, spaced apart by a dark field time interval having a selected length.

Some preferred frequencies of application for a time varying magnetic field are the following: (i) 1.7 Hz and/or 8 Hz (primarily for general stress reduction or relief); (ii) 4 Hz and/or 80 Hz (primarily for relief of sports-related stress); (iii) around 266 Hz (primarily for regeneration or cosmetic purposes); and/or (iv) other low frequencies suitable for stress relief, component regeneration and/or maintenance of beneficial chemical or physical reactions. For dental applications, the preferred frequencies of application are similar but further include a frequency of application around 666 Hz for regeneration. These treatments are normally applied for time intervals of 15-45 minutes but can be applied for shorter or longer time intervals as well. An acupuncture channel (meridian) may preferentially transport a magnetic field signal in somewhat the same manner that a light beam is believed to be preferentially transported by an acupuncture channel within a body.

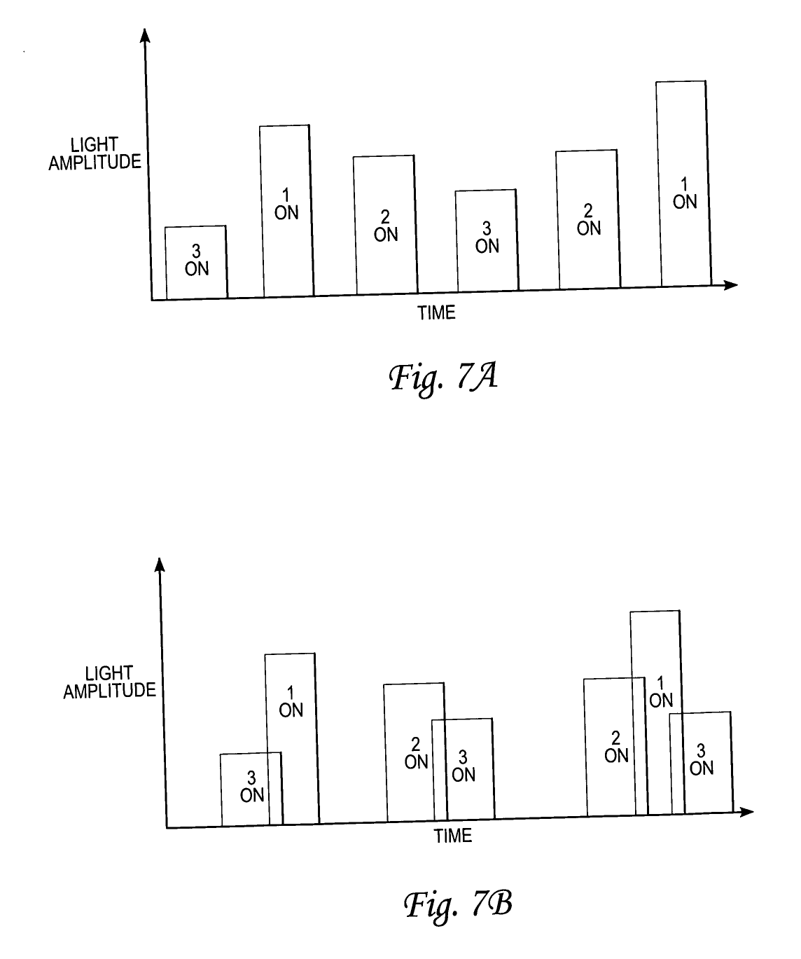

In a preferred embodiment of the invention, the light sources for the different wavelength ranges provide light in different time intervals, with a dark field time interval imposed between two consecutive irradiation time intervals for the same wavelength range. FIG. 7A is a graphical view of time intervals during which the first, second and third light sources (1), (2) and (3) are activated in a non-overlapping manner for different wavelength ranges. FIG. 7B is a graphical view of a second version, in which the light sources (1), (2) and (3) are activated in selected overlapping time intervals for different wavelength ranges. Preferably, two time intervals for delivery of the same wavelength range are spaced apart by a dark field time interval for that wavelength. More generally, N (≧1) sets of independently activatable light sources (N=1, 2 or 3 in FIGS. 7A and 7B) are provided, and N wavelength ranges are chosen within the near-ultraviolet, visible, near-infrared and mid-infrared wavelengths.

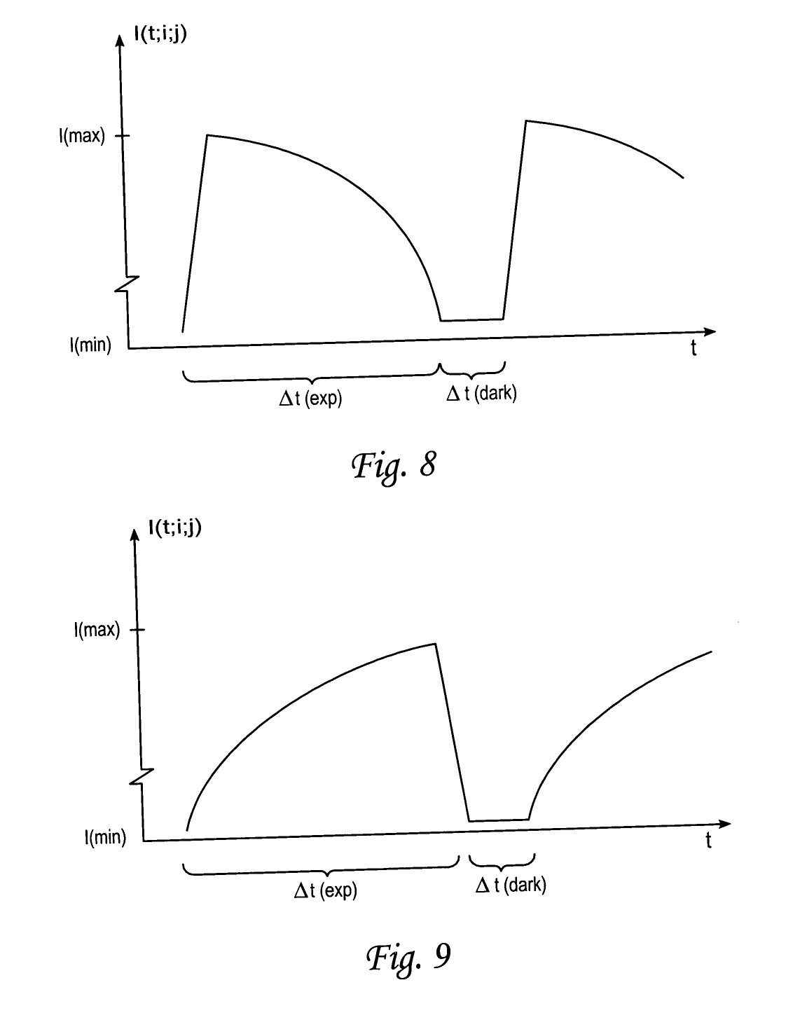

FIGS. 8, 9 and 10 illustrate examples of illumination intensity patterns of light activation (exposure interval) and deactivation (dark field interval) that can be used for the individual light elements 53(i,j) and/or for the activated magnetic field elements and/or the activated radio wave field elements in FIGS. 4, 5 and 6. In FIG. 8, the illumination intensity I(t;i;j) is substantially zero, then rises quickly to a maximum value I(max), then decreases monotonically to a lower value I(min) over an exposure time interval of length Δt(exp), remains at a small or substantially zero value for a dark field time interval of length Δt(dark), then optionally repeats this pattern at least once.

In FIG. 9, the illumination intensity I(t;i;j) rises monotonically from a substantially zero value to a maximum value I(max), then falls quickly to a small or substantially zero value I(min), over an exposure time interval of length Δt(exp), remains at a small or substantially zero value for a dark field time interval of length Δt(dark), then optionally repeats this pattern at least once.

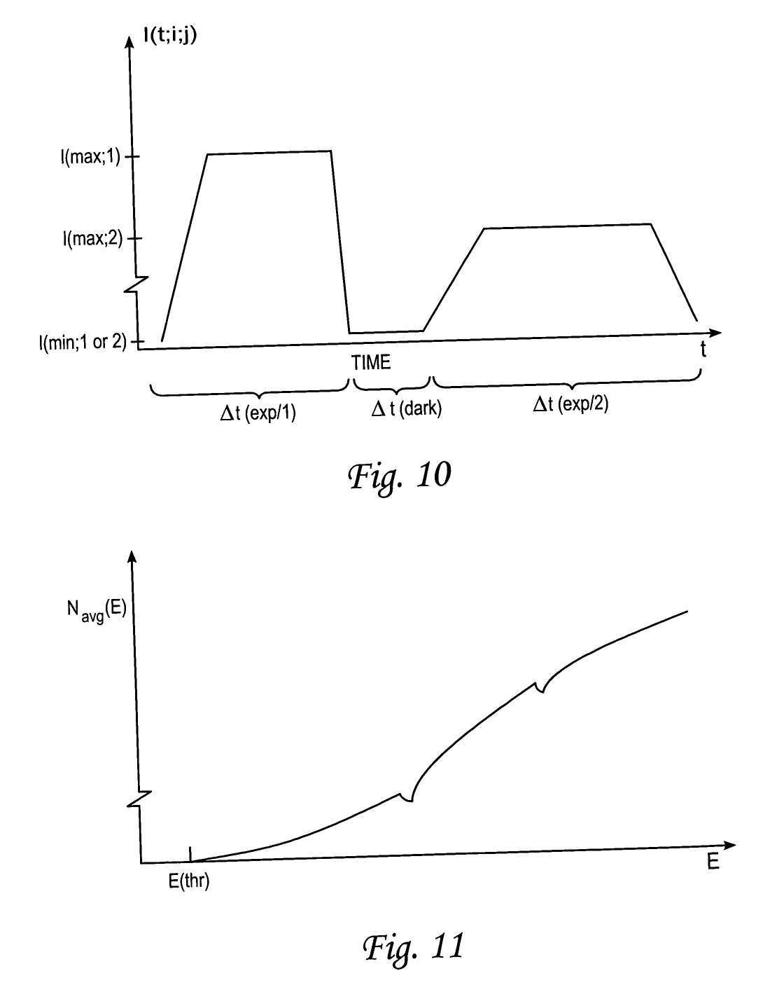

In FIG. 10, the illumination intensity I(t;i;j) rises to a first maximum value I(max; 1), optionally continues at or near that level for a first selected illumination time interval of length Δt(exp/1), falls to a first lower value I(min;1) that is substantially zero, remains at or near zero for a dark field time interval of length Δt(dark), rises to a second maximum value I(max;2), optionally continues at that level for a second selected illumination time interval of length Δt(exp/2), falls to a second lower value I(min;2) that is substantially zero, remains at or near zero, and optionally repeats this pattern. The maximum intensities I(max;1) and I(max;2) may be the same or may differ, the minimum intensities I(min;1) and I(min;2) may be the same or may differ, and one or both of the minimum intensities I(min;1) and I(min;2) may be 0. Light intensity patterns other than those shown in FIGS. 8, 9 and 10 can be used.

Each photon delivered to a vicinity of a body component 12 of the user in FIG. 1 is intended to produce one or more (preferably many) free electrons through photoelectric absorption and/or Compton scattering of the photon in its peregrinations through the body component and other body material. I have found, by analogy with the Einstein photoelectric effect in a metallic or crystalline material, that the photon energy E must be at least a threshold value E(thr), which lies in a range of about 0.8-3.1 eV, depending upon the atomic and/or molecular constituents of the selected body component and surrounding material, in order to produce at least one free electron as the photon undergoes scattering within the body. A photon with a wavelength λ=500 nm has an associated energy of 2.48 eV, for example. Not all photons with energies E just above the threshold value E(thr) will produce a free electron. A graph of average number N_{avg}(E) of free electrons produced for a given incident photon energy E might resemble the graph in FIG. 11. This graph is similar to a graph of average number of free electrons produced by a photon incident on a metallic or crystalline material according to the Einstein model.

Another important parameter is the rate r at which energy (or photons) is delivered to a unit area (e.g., over 1 cm²) of body surface per unit time (e.g., in 1 sec), during an exposure time interval. My experiments indicate that energy density rates r in a range 0.0013 Joules/cm²/sec≦r≦0.02 Joules/cm²/sec, averaged over a time interval of 5-45 min, is an appropriate range for many body components. Delivery of energy at a rate lower than about 0.0013 Joules/cm²/sec will have some effect but will require much longer radiation application times than a typical application time of 5-45 min. Delivery of energy at a rate greater than about 0.02 Joules/cm²/sec may saturate the body's ability to distribute the photon energy and may produce burns, ionization or other undesired local sensitization of the body. The peak light intensity I(t;i;j), shown in the examples of FIGS. 8, 9 and 10, will partly determine the energy delivery rate r.

Another important parameter is accumulated energy E(accum) delivered per unit area for the session in which radiation is applied. My experiments indicate that an accumulated energy density range of 2.5 Joules/cm²≦E(accum)≦20 Joules/cm² is an appropriate range for many body components.

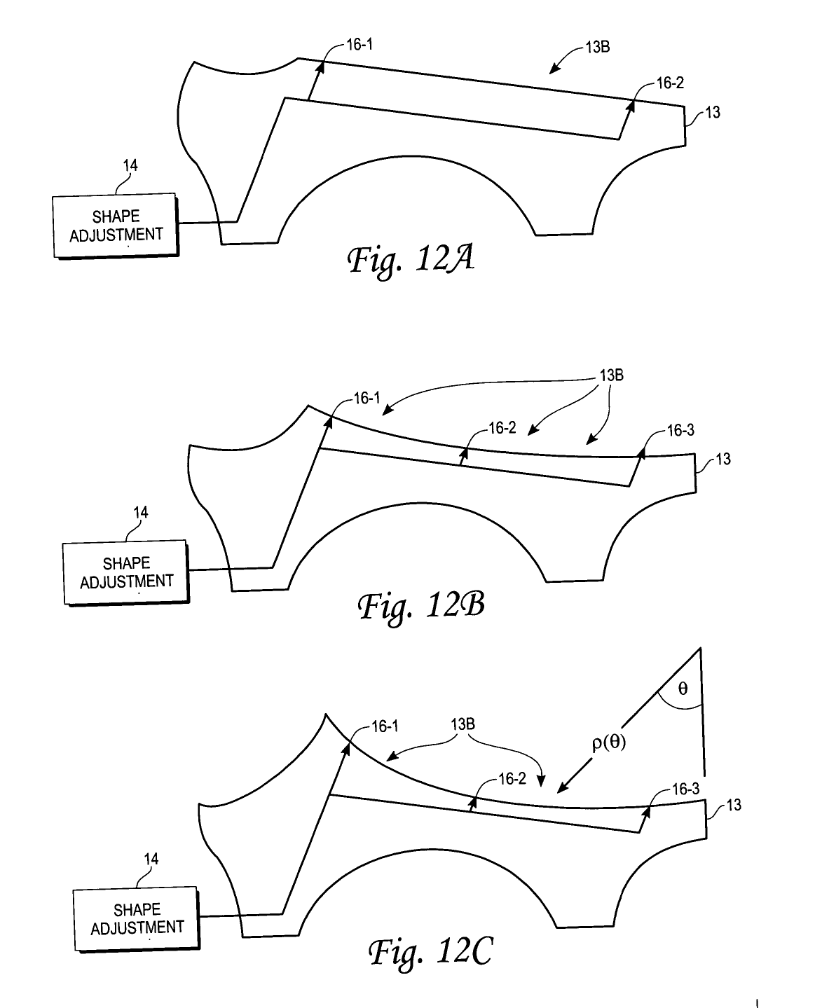

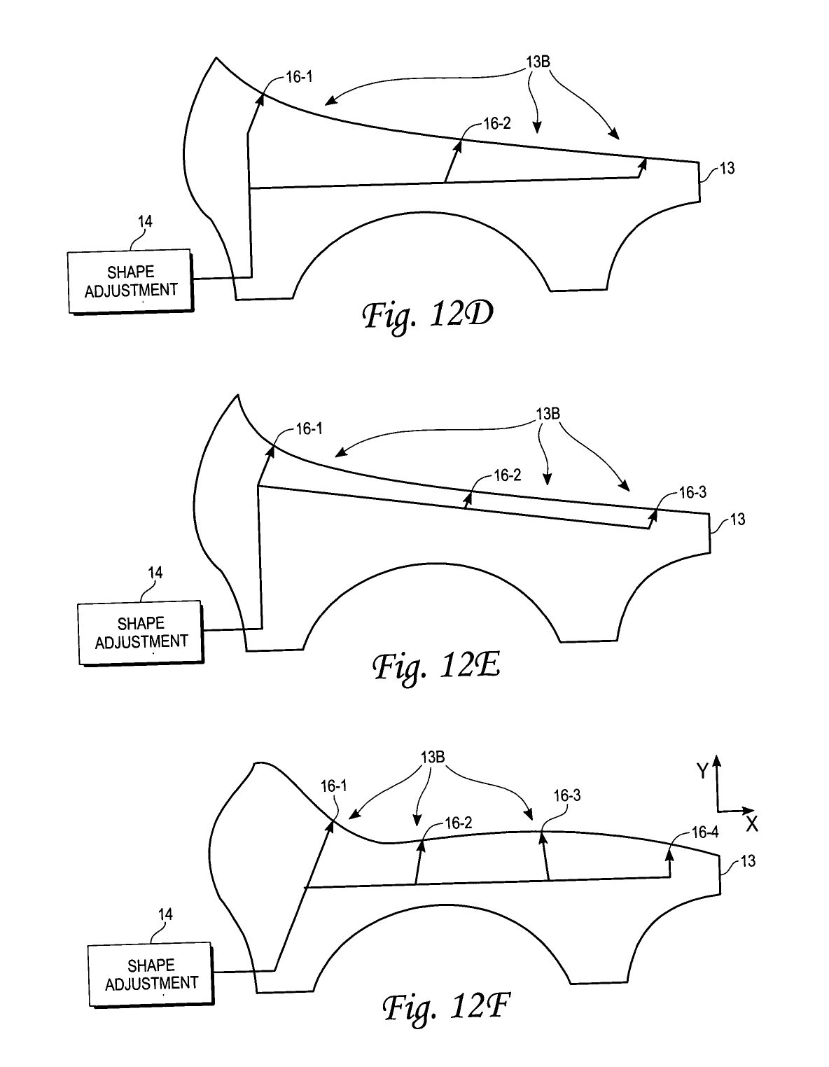

The cross-sectional shape of the body support module 13 may have a body support surface 13B with a cross-sectional shape that is linear or flat (horizontal or inclined), as shown in FIG. 12A, or is curvilinear, as illustrated in the examples shown in FIGS. 12B-12F. I have found that a user is more likely to relax if a curvilinear cross section is provided for the illumination session. Optionally, the cross-sectional shape can be changed with the passage of time, within a given session or from one session to the next session, by providing a shape adjustment mechanism 14 that controls the cross-sectional shape at two or more locations 16−k (k=1, . . . , K; K 2) on the body support surface 13B in FIGS. 12A-12F.

FIG. 12B illustrates a substantially circular cross-sectional shape in which the radius of curvature ρ is substantially constant along the body support surface 13B FIG. 12C illustrates a spiral cross-sectional shape, in which the radius of curvature ρ decreases monotonically as one moves toward a first (head) end of the body support surface 13B (e.g., ρ(θ)=a−b·θ, where a and b are positive coefficients and θ is an angle measured as indicated in FIG. 12C). FIG. 12D illustrates a first hybrid cross-sectional shape, including a circular portion at the first (head) end, augmented by a linear (flat) shape at a second (foot) end of the body support surface 13B. FIG. 12E illustrates a second hybrid cross-sectional shape, including a spiral portion at the first (head) end, augmented by a linear (flat) shape at a second (foot) end of the body support surface 13B. FIG. 12F illustrates a third hybrid cross-sectional shape, that may be described, for example, by an nth degree equation (e.g., y(x)=a₀+a₁·x+a₂·x²+ . . . +aₙ·xⁿ, (n≧2), where a₁, and aₙ are non-zero coefficients and x and y are measured as indicated in FIG. 12F. In some instances, the shape of the body support surface 13B may be changed one or more times within a session, or may be changed between sessions to accommodate the needs of the previous user and the present user.

The shape adjustment mechanism 14 may be incorporated in the control system, 22A or 22B or 22C in FIG. 1, and the control system may be programmed to automatically set one or more of the following in response to entry of a PIN number or another identification indicium: (1) the whole body or specified body components to be illuminated; (2) temporal length of the session; (3) the intensity(ies) of light to be delivered by each light source in the light delivery system; (4) the wavelength range(s) of light to be delivered to the whole body or to specified body components; (5) the exposure time Δt(exp) for each wavelength group used; (6) the dark time interval length Δt(dark) for each wavelength range used; (7) the light energy delivery rate to be delivered to the whole body or to specified body components; (8) the accumulated time light is to be delivered to the whole body or to specified body components; (9) the intensity(ies) of the magnetic field sources; (10) the frequency(ies) (including 0 Hz or dc) for the magnetic field sources; (11) the intensity(ies) of the LF frequency sources; (12) the frequency(ies) for the LF frequency sources; (13) the intensity(ies), radio frequency(ies) and time interval(s) of application of the radio waves; and (14) one or more shape parameters for the body support surface for one or more parts of a session.

The system can also accumulate and store information on the dates and lengths of sessions and/or number of sessions the user has engaged in over some selected time interval, such as the preceding one month, three months, six months or twelve months. Alternatively, part or all of this information may captured and stored on a user smart card that is passed through and read by the system before each user session.

The system disclosed here has the capability of restoring the function(s) of certain organs and tissues so that such an organ or tissue responds as if it were many years younger. To this extent, the system functions as a “time machine” to restore the responses of these organs and tissues to an earlier time.

1. A system for irradiating a human body, the system comprising:
a recliner that receives and supports at least a portion of a human body in a substantially horizontal body orientation on a recliner base support, the recliner having first and second ends and having a canopy, attached to and rotatable relative to the recliner first end;
an array of at least first, second and third spaced apart, individually activatable light sources, positioned on a surface of the recliner base support and on a surface of the canopy adjacent to the surface of the recliner base support, where the first, second and third light sources emit light in selected first, second and third wavelength ranges, respectively, drawn from the visible, infrared and near-ultraviolet spectra, where at least two of the first, second and third wavelength ranges may overlap with each other or may be spaced apart from each other;
a light source control mechanism, connected to each activatable light source and configured to activate the first light source during first and second spaced apart time intervals, to activate the second light source during third and fourth spaced apart time intervals, and to activate the third light source during fifth and sixth spaced apart time intervals;
at least first and second spaced apart, individually activatable low frequency (LF) wave sources, emitting a first LF wave and a second LF wave in a first LF frequency range and in a second LF frequency range, respectively, and being located on or adjacent to the recliner surface that receives the human's body; and
an LF source control mechanism, connected to the first and second LF wave sources and configured to activate the first LF wave source and the second LF wave source during a seventh time interval and an eighth time interval, respectively.

2. The system of claim 1, wherein at least one of said first, second and third wavelength ranges includes at least one of the wavelengths λ=470 nm, λ=550 nm, λ=637 nm,λ=666 nm, λ=890 nm and λ=905 nm.

3. The system of claim 1, wherein at least one of said first, second and third wavelength ranges is contained in a wavelength range 350 nm≦λ≦1500 nm.

4. The system of claim 1, wherein said first and said second wavelength ranges have substantially no wavelength overlap.

5. The system of claim 1, wherein at least one of said first, second and third light sources provides light having a wavelength in at least two of said first, said second and said third wavelength ranges.

6. The system of claim 1, wherein at least one of said first, second and third light sources provides light having a wavelength in all of said first, said second and said third wavelength ranges.

7. The system of claim 1, wherein said first and said second wavelength ranges are substantially the same.

8. The system of claim 1, wherein at least one of said third and said fourth time intervals overlaps with at least one of said first and said second time intervals.

9. The system of claim 1, wherein at least one of said third and said fourth time intervals does not overlap said first time interval and does not overlap said second time interval.

10. The system of claim 1, wherein said light source control mechanism provides at least one dark field time interval, having at least a selected positive temporal length Δt(dark), that does not overlap any of said first, said second, said third, said fourth, said fifth and said sixth time intervals.

11. The system of claim 10, wherein said dark field time interval length satisfies 0.1 sec≦Δt(dark)≦1 sec.

12. The system of claim 1, wherein at least one of said first, said second and said third light sources, when activated, provides said light having an energy delivery rate r lying in a range 0.0013 Joules/cm²/sec≦r≦0.02 Joules/cm²/sec.

13. The system of claim 1, wherein at least one of said first, said second and said third light sources, when activated, provides said light to a selected portion of said human body having an accumulated energy density E(accum) lying in a range 2.5 Joules/cm²≦E(accum)≦20 Joules/cm².

14. The system of claim 1, wherein at least one of said LF wave frequencies f(LF) is drawn from a range 1 Hz≦f(LF)≦10⁴ Hz.

15. The system of claim 1, wherein said seventh time interval overlaps at said eighth time interval.

16. The system of claim 1, wherein said first and second LF frequency ranges are substantially the same.

17. The system of claim 1, wherin said first and second LF frequency ranges have substantially no frequency overlap.

18. The system of claim 1, further comprising:
first, second and third activatable magnetic field sources, located adjacent to said first, said second and said third light sources, respectively, with each of the magnetic field sources having a magnetic field intensity B substantially in a range 100 Gauss≦B≦10⁴ Gauss; and
a magnetic field source control mechanism, connected to the first, second and third magnetic field sources and configured to activate the first magnetic field source, the second magnetic field source and the third magnetic field source during a ninth time interval, a tenth time interval and an eleventh time interval, respectively.

19. The system of claim 18, wherein at least one of said first, said second and said third magnetic field source provides a time varying magnetic field having at least one magnetic field frequency f(mag) in a range 1 Hz≦f(mag)≦10⁴ Hz in at least one magnetic field direction.

20. The system of claim 18, wherein each of said first, said second and said third magnetic field source provides a substantially constant magnetic field in at least one magnetic field direction.

21. The system of claim 1, wherein said canopy rotates to a first canopy position in which at least a portion of a head of said human's body lies between said canopy and said recliner first end, and to a second canopy position that allows said recliner base support to receive said human body.

22. The system of claim 1, wherein said recliner base support has a cross sectional shape drawn from the following group of selected shapes: (i) linear or flat; (ii) a sector of a circle; (iii) a portion of a spiral; (iv) a sector of a circle, augmented by a linear section; (v) a portion of a spiral, augmented by a linear section; and (vi) a shape substantially described by an nth degree equation, with n≧2.

23. The system of claim 22, further comprising a cross sectional shape adjustment mechanism, attached to said recliner base support at at least two locations, to implement a change of cross sectional shape of said recliner base support from one of said group of selected shapes to another of said group of selected shapes.

24. The system of claim 1, wherein said light control mechanism controls at least one of the following parameters associated with application of the system to said human's body during at least one continuous application session: (1) a whole body or at least one specified body component to be illuminated; (2) temporal length of the session; (3) an intensity of said light to be delivered by at least one of said light sources; (4) at least one wavelength range of said light to be delivered to the whole body or to the at least one specified body component; (5) an exposure time Δt(exp) for the at least one wavelength range used; (6) a dark time interval length Δt(dark) for the at least one wavelength range used; (7) a light energy delivery rate for the whole body or for the at least one specified body component; (8) an accumulated time light is to be delivered to the whole body or to the at least one specified body component; (9) an intensity of a magnetic field applied to said body; (10) a frequency (including 0 Hz) for the magnetic field applied to said body; (11) an intensity of at least one of said LF frequency sources; (12) a frequency for the at least one LF frequency source; (13) an intensity, a radio frequency and and a time interval of application of a radio wave applied to said body; and (14) one or more shape parameters that define a shape for said recliner body support for at least one time interval for the session.

25. A method for irradiating a human body, the method comprising:
positioning at least a portion of a human body on a recliner that supports the body in a substantially horizontal body orientation on a recliner base support, the recliner having first and second ends and having a canopy, attached to and rotatable relative to the recliner first end;
providing an array of at least first, second and third spaced apart, individual light sources, positioned on a surface of the recliner base support and on a surface of the canopy adjacent to the surface of the recliner base support, where the first, second and third light sources emit light in selected first, second and third wavelength ranges, respectively, drawn from the visible, infrared and near-ultraviolet spectra, where at least two of the first, second and third wavelength ranges may overlap with each other or may be spaced apart from each other;
activating the first light source during first and second spaced apart time intervals, activating the second light source during third and fourth spaced apart time intervals, and activating the third light source during fifth and sixth spaced apart time intervals;
providing at least first and second spaced apart, individual low frequency (LF) wave sources, which emit a first LF wave and a second LF wave in a first LF frequency range and in a second LF frequency range, respectively, where the LF wave sources are located on or adjacent to the recliner surface that receives the human's body; and
activating the first LF wave source and the second LF wave source during a seventh time interval and an eighth time interval, respectively.

26. The method of claim 25, further comprising choosing at least one of said first, second and third wavelength ranges to include at least one of the wavelengths λ=470 nm, λ=550 nm, λ=637 nm, λ=666 nm, λ=890 nm and λ=905 nm.

27. The method of claim 25, further comprising choosing at least one of said first, second and third wavelength ranges to be contained in a wavelength range 350 nm≦λ≦1500 nm.

28. The method of claim 25, further comprising providing, from at least one of said first, second and third light sources, light having a wavelength in at least two of said first, said second and said third wavelength ranges.

29. The method of claim 25, further comprising providing, from at least one of said first, second and third light sources, light having a wavelength in all of said first, said second and said third wavelength ranges.

30. The method of claim 25, further comprising providing at least one dark field time interval, having at least a selected positive temporal length Δt(dark), that does not overlap any of said first, said second, said third, said fourth, said fifth and said sixth time intervals.

31. The method of claim 25, further comprising:
providing first, second and third activatable magnetic field sources, located adjacent to said first, said second and said third light sources, respectively, with each of the magnetic field sources having a magnetic field intensity B substantially in a range 100 Gauss≦B≦10⁴ Gauss; and
activating the first magnetic field source, the second magnetic field source and the third magnetic field source during a ninth time interval, a tenth time interval and an eleventh time interval, respectively.

32. The method of claim 25, further comprising providing said recliner base support with a cross sectional shape drawn from the following group of shapes: (i) linear or flat; (ii) a sector of a circle; (iii) a portion of a spiral; (iv) a sector of a circle, augmented by a linear section; (v) a portion of a spiral, augmented by a linear section; and (vi) a shape substantially described by an nth degree equation, with n≧2.

33. The method of claim 32, further comprising changing said cross sectional shape to another of said shapes drawn from said group of shapes (i), (ii), (iii), (iv), (v) and (vi) to another of said group of shapes (i), (ii), (iii), (iv), (v) and (vi).

34. The method of claim 25, further comprising controlling at least one of the following parameters associated with application of the system to said human's body during at least one continuous application session: (1) a whole body or at least one specified body component to be illuminated; (2) temporal length of the session; (3) an intensity of said light to be delivered by at least one of said light sources; (4) at least one wavelength range of said light to be delivered to the whole body or to the at least one specified body component; (5) an exposure time Δt(exp) for the at least one wavelength range used; (6) a dark time interval length Δt(dark) for the at least one wavelength range used; (7) a light energy delivery rate for the whole body or for the at least one specified body component; (8) an accumulated time light is to be delivered to the whole body or to the least one specified body component; (9) an intensity of a magnetic field applied to said body; (10) a frequency (including 0 Hz) for the magnetic field applied to said body; (11) an intensity of at least one of said LF frequency sources; (12) a frequency for the at least one LF frequency source; (13) an intensity, a radio frequency and and a time interval of application of a radio wave applied to said body; and (14) one or more shape parameters that define a shape for said recliner body support for at least one time interval for the session.
